(19) United States
(12) Patent Application Publication
(10) Pub. No.: US 2012/0004501 A1
(43) Pub. Date: Jan. 5, 2012

(54) Method and Apparatus for Prolapse Repair

(75) Inventor: Roger D. Beyer, Paw Paw, MI (US)

(73) Assignee: AMS Research Corporation, Minnetonka, MN (US)

(21) Appl. No.: 13/256,185

(22) PCT Filed: Mar. 12, 2010
(86) PCT No.: PCT/US10/27154

§ 371 Date: Sep. 12, 2011

Related U.S. Application Data
(60) Provisional application No. 61/159,719, filed on Mar. 12, 2009.

(51) Int. Cl. A61F 2/00

(52) U.S. Cl. 600/37

(57) ABSTRACT

A surgical support apparatus and method includes a central support member or portion and six or more straps or arms, with each of the straps comprising a connector configured to mate with a tip of a needle device. Each connector is adapted for attachment to target tissue within the pelvis of a patient such that attachment of the connectors to the patient tissue allows for selective placement of the central support member to provide internal pelvic support, e.g., treat vaginal prolapse. Further, a neo-ligament apparatus can be provided. The neo-ligament apparatus generally includes a first member, and a second member connected to and extending out from first member at a junction, at a predetermined angle.

### RELATED APPLICATION

This application claims priority to and the benefit of U.S. Provisional Application No. 61/159,719, filed Mar. 12, 2009, which is incorporated herein by reference in its entirety.

### FIELD OF THE INVENTION

The present invention relates generally to urogenital surgery and, more specifically, to repairing genital prolapse in a patient.

### BACKGROUND OF THE INVENTION

Female genital prolapse has long plagued women. Vaginal prolapse develops when intra-abdominal pressure pushes the vagina outside the body. In a normal situation, the levator ani muscles close the pelvic floor. This results in little force being applied to the fasciae and ligaments that support the genital organs. Increases in abdominal pressure, failure of the muscles to keep the pelvic floor closed, and damage to the ligaments and fasciae all contribute to the development of prolapse.

Many techniques have been tried to correct or ameliorate the prolapse and its symptoms, with varying degrees of success. Nonsurgical treatment of prolapse involves measures to improve the factors associated with prolapse, including treating chronic cough, obesity, and constipation. Other nonsurgical treatments may include pelvic muscles exercises or supplementation with estrogen. These therapies may alleviate symptoms and prevent worsening, but the actual hernia or weakness will remain. Vaginal pessaries are the primary type of nonsurgical treatment, but there can be complications due to vaginal wall ulceration.

There is a desire to obtain a minimally invasive yet highly effective device and method that can be used to treat pelvic organ prolapse with minimal to no side effects. Such a device should increase organ support, reduce the complexity of the surgical procedure, be biocompatible, adjustable, and non-toxic. The treatment methods using the device should reduce pain, operative risks, infections and post operative hospital stays. Further, the method of treatment should also improve the quality of life for patients.

### SUMMARY OF THE INVENTION

The present invention broadly provides a method and apparatus for genital prolapse repair. In one embodiment, the method includes the steps of: establishing a plurality of pathways in tissue around a bladder of a patient, introducing a strap or arm into each of the pathways, and positioning beneath the bladder of the patient a support member having each of the straps connected thereto such that the bladder of the patient is supported by the support member. A bulge of the bladder into a vagina of the patient is reduced as a consequence of applying this method.

In one embodiment, a surgical implant kit includes a support apparatus including a central support member and at least six straps or arms, each of the straps comprising a connector configured to mate with a tip of a needle. Each connector is adapted for attachment to or penetration through tissue within the pelvic region of a patient such that attachment of the connectors to the patient tissue allows for selective placement of the central support member to support the patient's bladder or other anatomical structures. The kit can further include a plurality of needle devices, adapted to engage and direct the connectors of the apparatus.

In another embodiment, a surgical implant kit includes a support apparatus including a central support member and at least eight straps or arms, each of the straps comprising a connector configured to mate with a tip of a needle. Each connector is adapted for attachment to tissue within the pelvic region of a patient such that attachment of the connectors to the patient tissue allows for selective placement of the central support member to support the patient's bladder.

In yet another embodiment, the connector and/or the straps of a support apparatus include indicia matching indicia on a corresponding needle device.

In another embodiment, a neo-ligament apparatus is provided that generally includes a first strap or arm member, and a second strap or arm member connected to and extending out from the first member. The second member can extend out from the first member at an angle, such as approximately 45 degrees. Each of the members can include end connectors or anchors adapted for attachment to target tissue within the patient such that the neo-ligament apparatus can be employed to repair levator ballooning, vaginal prolapse, and/or rectal prolapse.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a fragmented front schematic view of a surgical support apparatus with connecting tabs and attachable connecting straps in accordance with embodiments of the present invention.

FIG. 2 is a fragmented front schematic view of a surgical support apparatus with integral connecting straps in accordance with embodiments of the present invention.

FIG. 3 is a fragmented front schematic view of a surgical support apparatus with attachable connecting straps in accordance with embodiments of the present invention.

FIG. 4 is a fragmented front schematic view of a neo-ligament apparatus in accordance with embodiments of the present invention.

FIG. 5 is a front schematic view of a neo-ligament apparatus with intermediate mesh support in accordance with embodiments of the present invention.

### DETAILED DESCRIPTION OF THE PREFERRED EMBODIMENTS

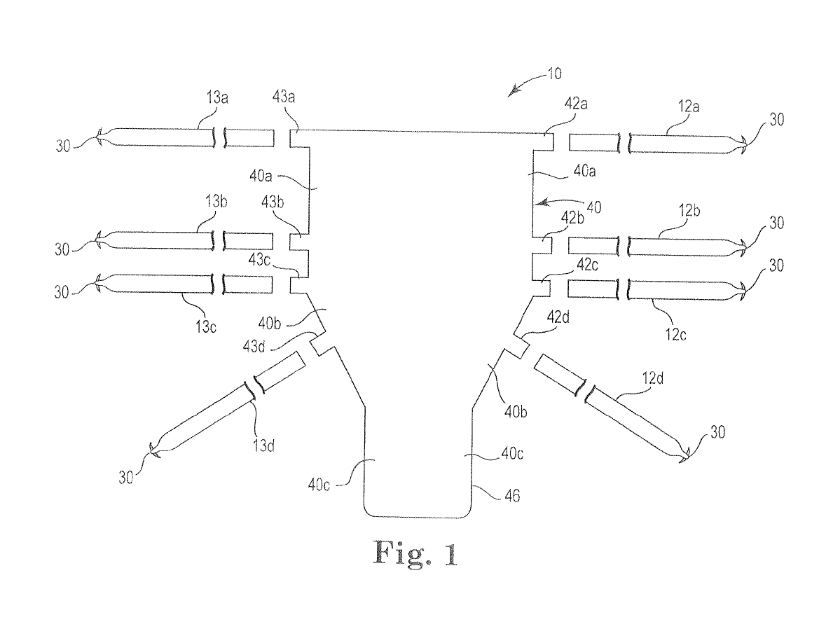

Referring generally to FIGS. 1-3, a surgical support apparatus or implant 10 according to embodiments of the present invention is shown. The apparatus 10 can be configured and adapted to be surgically implanted in a female patient to repair anterior prolapse of the vagina. Further, the present invention may be used to correct central defects, midline defects, or both midline and central defects at once.

Apparatus 10 can comprise four straps or arms 12a-12d, four straps or arms 13a-13d, and a support member or portion 40. In such an embodiment, a total of eight straps 12, 13 are provided and can correspond with superior and inferior anatomical positioning and attachment. Straps 12a, 13a can be opposingly provided to extend at or proximate an end region of the support member 40, with straps 12b,c and 13b,c positioned to extend at an intermediate or mid region of the support member 40, and straps 12d, 13d extending from a tail or second end region of the support member 40. Such strap mirroring generally provides bilateral attachment or support.

In other embodiments, a total of six straps 12, 13 can be included with apparatus 10. For instance, rather than providing two straps extending from each side of the support member mid region 40b, a single strap could be included on each side at the mid region. Other embodiments are envisioned as well with varying combinations, numbers and configurations for providing the straps along the support member 40.

Each of straps 12a-13d and 13a-13d can include a connector or anchor 30 at its respective distal end. Further, each strap 12a-12d and 13a-13d can be covered by a respective sheath (not shown). The anchors 30 are adapted to attach to or penetrate through at least a portion of anatomical structure or tissue within the pelvic region of the patient. Certain embodiments of the anchors 30 can include one or more tines or barbs provided to facilitate tissue penetration and attachment.

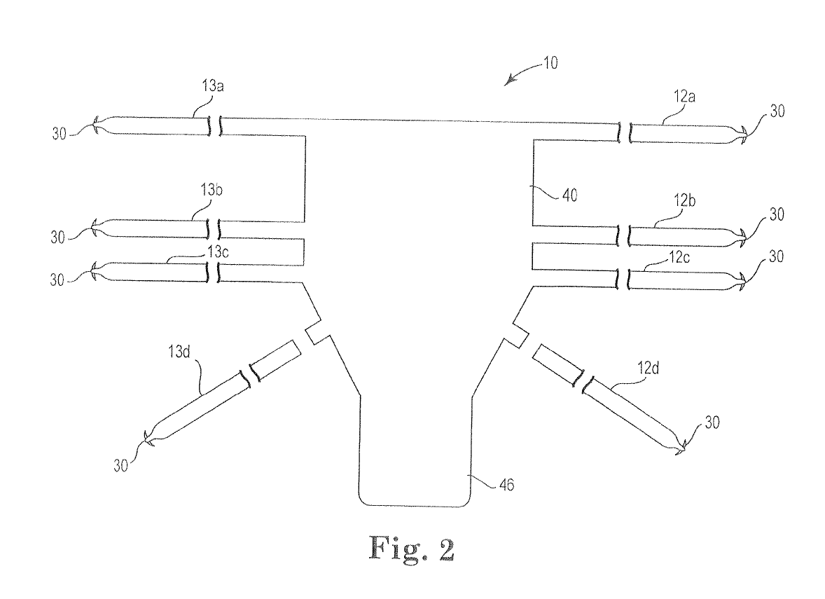

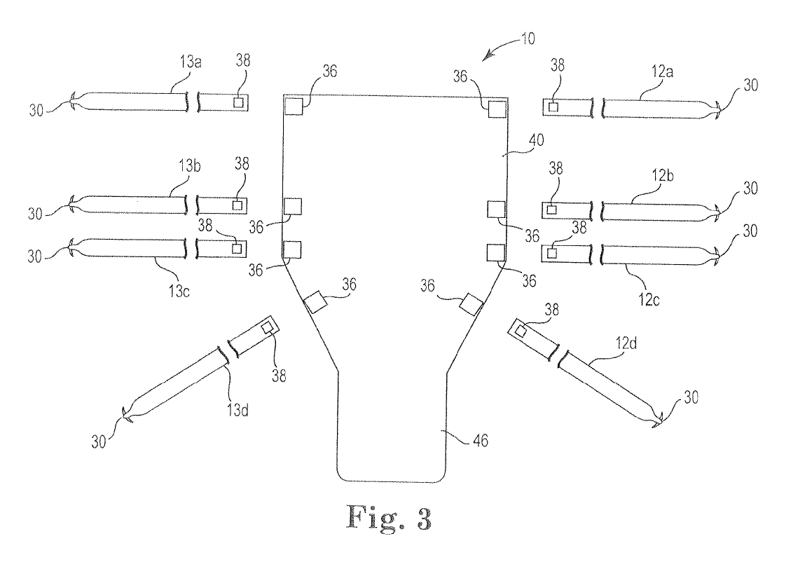

Straps 12a-12d and 13a-13d can be constructed of known compatible mesh material and can be connected to tabs 42a-42d and 43a-43d of support member 40, respectively, by known means, as shown in FIG. 1. Alternatively, one or all of straps 12a-12d and 13a-13d can be integrally formed with support member 40, as shown in FIG. 2. In yet another embodiment, the straps 12a-12d and 13a-13d can be connected to support member 40 by linking portions 36 of support member 40 with portions 38 of straps 12a-12d, 13a-13d, as shown in FIG. 3. With such an embodiment, clips, rivets, rings, eyelets, grommets, or other mechanisms or devices known in the art can be used to attach portions 36, 38. The positional attachment of straps or arms 12, 13 to tissue can vary depending on the particular support requirements and respective anatomy of the patient.

In one embodiment, sheath is made of polyethylene. Other material may be used, such as polypropylene, nylon, polyester, or Teflon™. The sheath is configured for removal from the strap after the strap is in the correct position in the body. In general, the sheath can be employed to facilitate deployment of the apparatus, providing needed stiffness, tissue protection, or like benefits.

Straps 12a-12d and 13a-13d, and/or respective sheaths, may also include indicia provided thereon to signify the correct orientation for implantation into a patient. For instance, sheaths can include indicia to identify the respective straps 12 or 13 as superior or inferior straps during deployment, adjustment and general manipulation of the apparatus 10. Words, symbols, and colors are all possible indicia that may be used, and these modifications are intended to be within the spirit and scope of the invention as claimed. Further, the indicia may be located on any or all of the straps, the connectors, and the sheaths.

Suitable connectors or anchors, deployment tools, materials, devices and implant components envisioned for use with the present invention are disclosed in U.S. Pat. No. 7,351,197, International Patent Publication Nos. WO 2008/057261 and WO 2007/097994, and U.S. Patent Publication Nos. 2009/0192347, 2002/151762 and 2002/147382. Accordingly, the above-identified disclosures are hereby incorporated by reference in their entirety.

In one embodiment, connectors 30 of each of the straps 12, 13 are configured to attach generally at eight respective tissue targets. As such, mirroring anatomical support connections (e.g., superior and inferior) are provided in pairing straps 12a and 13a, 12b and 13b, 12c and 13c, and 12d and 13d. For example, straps 12a, 13a can extend such that their connectors 30 are attachable at or proximate the obturator internus or levator muscles; straps 12b, 13b can be attachable at or proximate the “white line”; and straps 12c-d, 13c-d can be attachable at or proximate the uterosacral/cardinal ligament complex.

Support member 40 can be sized and shaped to afford repair of a cystocele without directly lifting the patient's bladder and placing undue tension on the bladder or vaginal wall. Support member 40 can be constructed of various known materials utilized in repairing vaginal prolapse, e.g., compatible mesh or polymer materials. The shape of member 40 may be predetermined, or the member may be trimmed based on patient anatomy before implantation. For instance, support member 40 can include a tail portion 46 at end region 40c that can be trimmed according to the anatomy of the patient to prevent bunching. Similarly, the size and shape of support member 40 (e.g., regions 40a-40c), straps 12, 13, and connectors 30 can vary widely depending on manufacturing and vaginal support objectives, as well as the anatomy of the particular patient. The final form of support member 40 can be manipulated during implantation based generally on its material and shape construct, and the attachment of straps 12, 13. Further, various support attachment devices, sutures, material specifications, kit components, structures and other devices, methods and apparatus disclosed in the previously-incorporated references can be employed with the embodiments of the present invention. As shown in specific embodiments, the support member 40 can be generally wider at one end portion 40a, with a degree of tapering at the mid region 40b or opposing end 40c, to facilitate support, conformity, physical manipulation or overall performance of the implant 10. For instance, as shown in the embodiments of FIGS. 1-3, the mid region 40b can be angled or tapered such that it is narrower at least along a portion of its length compared to the top end region 40a. Further, the bottom or opposing end region 40e can extend from the mid region 40b to define the tail portion. The tail portion can be configured to have varying lengths depending on the particular application. The tail portion can be generally uniform for its width or it can taper or angle much like the previously-described mid region.

Surgical support apparatus 10 can include a biological graft or synthetic body for support member 40. To attach support member 40 to straps 12a-12d and 13a-13d, clamps, rivets, sutures, rings, eyelets, grommets, or other known connecting devices or techniques, can be used to hold and secure the surfaces of the strap and member together.

In various embodiments, support member 40 or straps 12, 13 can be constructed in whole, or in part, of a synthetic material, including polypropylene, cellulose, polyvinyl, silicone, polytetrafluoroethylene, polygalactin, Silastic, carbon-fiber, polyethylene, nylon, polyester (e.g., Dacron) polyanhydrides, polycaprolactone, polyglycolic acid, poly-L-lactic acid, poly-D-L-lactic acid and polyphosphate esters. Commercial examples of suitable synthetic materials include Marlex™ (polypropylene) available from Bard of Covington, R.I., Prolene™ (polypropylene); Prolene Soft Polypropylene Mesh or Gynemesh (nonabsorbable synthetic surgical mesh), both available from Ethicon, of New Jersey, and Mersilene (polyethylene terphthalate); Hernia Mesh also available from Ethicon, Gore-Tex™ (expanded polytetrafluoroethylene) available from W. L. Gore and Associates, Phoenix, Ariz.; the polymer supports available in the APOGEE™ PERIGEE™, or SPARC™ implant systems, available from American Medical Systems, Inc. of Minnetonka, Minn.; Dexon™ (polyglycolic acid) available from Davis and Geck of Danbury, Conn.; and Vicryl™ available from Ethicon. Other suitable synthetic materials known to those of ordinary skill in the art may be employed without deviating from the spirit and scope of the present invention.

In certain embodiments, support member 40 or straps 12, 13 may be constructed in whole, or in part, of a non-synthetic material. Suitable non-synthetic materials include allografts, homografts, heterografts, autologous tissues, cadaveric fascia, autodermal grafts, dermal collagen grafts, autofascial heterografts, whole skin grafts, porcine dermal collagen, lyophilized aortic homografts, preserved dural homografts, bovine pericardium and fascia lata. Other suitable non-synthetic materials known to those of ordinary skill in the art may be employed without deviating from the spirit and scope of the present invention.

The individual components or elements of the present invention may be packaged together in a kit assembly, including introduction or deployment devices or systems. Alternatively, the individual elements may be separately packaged or packaged in subassemblies depending on a variety of factors such as shelf life and sterilization requirements. They may be assembled at the manufacturing location or at the healthcare location. Any suitable sterilization procedure may be utilized to sterilize the contents of a kit. Suitable sterilization techniques include, but are not limited to, steam, ethylene oxide, electron beam, vapor (e.g., hydrogen peroxide or peracetic acid), gamma or plasma procedures.

In preparation for surgery, the patient can be placed in a modified dorsal lithotomic position with hips flexed, legs elevated in stirrups and buttocks even with the edge of the surgical table. The patient's bladder is emptied. A catheter is not required during the procedure, but may aid in identifying the urethra during the procedure. A weighted vaginal retractor or other suitable vaginal retraction can be used, if desired. Various techniques, devices and procedures known to those of ordinary skill in the art, and those disclosed in previously-incorporated U.S. Pat. No. 7,351,197, and International Patent Publication Nos. WO 2008/057261 and WO 2007/097994, are available for positioning and attaching the support member 40 and its corresponding straps 12, 13 and connectors 30 within the patient.

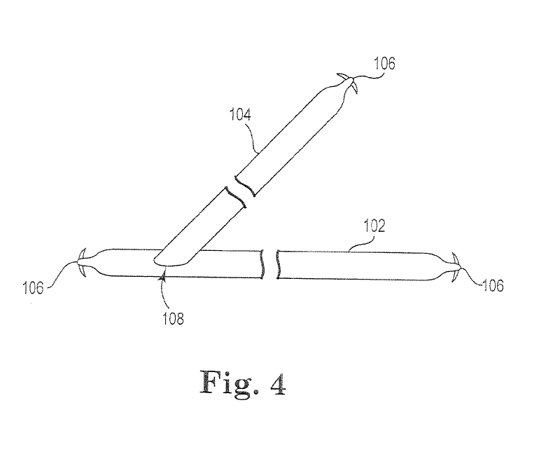

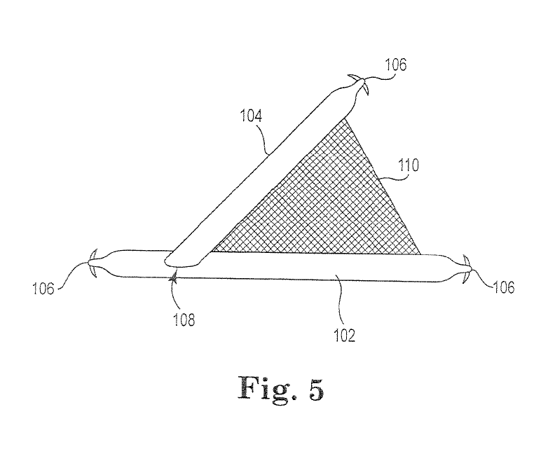

Referring generally to FIGS. 4-5, a neo-ligament apparatus or implant 100 in accordance with embodiments of the present invention is provided. The apparatus 100 generally includes a first strap or arm member 102, and a second strap or arm member 104 connected to and extending out from first member 102. The first and second members 102 and 104 can be integrally formed, or attached using known connection methods and techniques. In one embodiment, second member 104 extends out from first member 102 at an angle, such as approximately 45 degrees. Each of the members 102, 104 can include end connectors or anchors 106 adapted for attachment to target tissue within the patient. Each element or member of apparatus 100 can be constructed of materials, and configured with methods and techniques known to those of ordinary skill in the art, and as described herein or otherwise incorporated in the disclosures provided hereinabove. The neo-ligament apparatus 100 can be generally employed to repair levator ballooning, vaginal prolapse, rectal prolapse, and/or anal incontinence. As such, the apparatus 100 can replace or otherwise supplement anatomical structure within the pelvic region of the patient.

In one embodiment, the apparatus 100 includes a juncture 108, where an end portion of the second member 104 crosses or links with the first member 102. This junction 108 can emulate the anatomical juncture of the arcus tendineus rectovaginalis (posterior white line) with the arcus tendineus fasciae pelvis (anterior white line). As such, first member 102 can emulate the anterior white line and second member 104 can emulate the posterior white line to provide pelvic support to repair ballooning of the levators through ligament reconstruction. The connectors 106 can be respectively configured for attachment to the tissue structure where the posterior and anterior white lines attach, such as the perineal body and the ischial spine.

Further, first member 102 can include a plurality of optional, or selectively adjustable, connection points for second member 104 to create various junctures 108 along the length of first member 102. This permits adjustment of the angle and positioning of the second member 104 relative to the first member 102 to accommodate the specific anatomy of a particular patient and the reconstructing support requirements of the patient. Further, various pins, rivets or other devices or connectors can be provided at the juncture 108 to facilitate rotation, movement and overall adjustability.

In an alternative embodiment, apparatus 100 can further include an intermediate mesh portion 110. The mesh portion 110 can provide a netting support extending from the first member 102 to the angled second member 104, as shown in FIG. 5. The material construction, size, shape and positioning of the mesh portion 110 can vary widely depending on support requirements and the particular anatomy of the patient. In addition to those anatomical applications described, the apparatus 100 can be employed to provide neo-ligament or tissue support or repair for or along other anatomical structures within the pelvic region of the patient.

All patents, patent applications, and publications cited herein are hereby incorporated by reference in their entirety as if individually incorporated, and include those references incorporated within the identified patents, patent applications and publications.

Obviously, numerous modifications and variations of the present invention are possible in light of the above teachings. It is therefore to be understood that within the scope of the appended claims, the invention may be practiced other than as specifically described herein.

1. An implant apparatus for treatment of vaginal prolapse in a patient, comprising:
a support portion having a first end region, a mid region and a second end region, with the second end region provided generally narrower than the first end region;
a plurality of extending arms, including;
a first set of arms extending from respective sides of the first end region of the support portion;
a second set of arms extending from respective sides of the mid region of the support portion; and
a third set of arms extending from respective sides of the second end region of the support portion.

2. The apparatus of claim 1, further including an anchor provided with at least one of the plurality of extending arms.

3. The apparatus of claim 1, further including an anchor provided with each of the plurality of extending arms.

4. The apparatus of claim 1, wherein the plurality of arms are each integrally formed with the support portion.

5. The apparatus of claim 1, wherein the plurality of arms are each separately connectable with the support portion.

6. The apparatus of claim 1, further including at least one sheath provided to cover at least one of the plurality of arms.

7. The apparatus of claim 6, wherein the sheath is selectively removable from the at least one of the plurality of arms.

8. The apparatus of claim 1, further including an additional set of arms extending from respective sides of the mid region of the support portion.

9. The apparatus of claim 1, wherein the mid region of the support member is generally tapered such that it narrows in width toward the second end region.

10. The apparatus of claim 9, wherein the second set of arms extend generally transverse from the tapered mid region.

11. The apparatus of claim 1, wherein the first set of arms are adapted to operably attach to the obturator internus or levator muscles.

12. The apparatus of claim 1, wherein the second set of arms are adapted to operably attach to a portion of the white line of the patient.

13. The apparatus of claim 1, wherein the third set of arms are adapted to operably attach to the uterosacral ligament complex of the patient.

14. The apparatus of claim 1, wherein the support portion is constructed at least in part of a polymer mesh material.

15. A pelvic support implant for the treatment of vaginal prolapse in a patient, comprising:
a first end region having a generally uniform width;
a mid region having a generally tapered width;
a second end region generally narrower than the width of the first end region such that the mid region is in operable communication with the first end region and the second end region; and
at least a first arm operably extending from a first side of the tapered mid region, and at least a second arm operably extending from a second side of the tapered mid region, with each of the first and second arms including a distal tissue anchor.

16. The implant of claim 15, wherein the first and second arms extend generally transverse from the tapered mid region.

17. The implant of claim 15, wherein the first arm and second arm are adapted to operably attach to the white line to provide bilateral attachment for the support implant.

18. The implant of claim 15, wherein at least the mid region is constructed of a polymer mesh material.

19. A neo-ligament support apparatus for implantation within the pelvic region of a patient, comprising:
a first elongate arm member having at least one end anchor;
a second arm member extending from the first elongate arm member at an angle less than ninety degrees, such that a juncture is defined at the region of communication between the first elongate arm member and the second arm member.

20. The apparatus of claim 19, further including an intermediate support portion extending from the first elongate arm member to the second arm member.

21. The apparatus of claim 20, wherein the intermediate support portion is constructed at least in part of a polymer mesh material.

22. The apparatus of claim 19, wherein the juncture is positioned within the patient to generally emulate the anatomical juncture of the posterior white line with the anterior white line to treat levator ballooning.
